(19) United States
(12) Patent Application Publication
(10) Pub. No.: US 2023/0009401 A1
(43) Pub. Date: Jan. 12, 2023

(54) HIGH FREQUENCY COIL APPARATUS FOR OBTAINING NUCLEAR MAGNETIC RESONANCE SIGNALS OF OTHER NUCLIDES WITHIN MAGNETIC RESONANCE IMAGING SYSTEM, AND METHOD FOR OPERATING SAME

(71) Applicant: KOREA UNIVERSITY RESEARCH AND BUSINESS FOUNDATION, SEJONG CAMPUS, Sejong (KR)

(72) Inventors: Changhyun OH, Sejong (KR); Junsik YOON, Sejong (KR); Jongmin KIM, Sejong (KR); Gwangwoo JEONG, Sejong (KR)

(21) Appl. No.: 17/782,700

(22) PCT Filed: Nov. 18, 2020
(86) PCT No.: PCT/KR2020/016232

§ 371 Date: Jun. 6, 2022

(30) Foreign Application Priority Data
Dec. 4, 2019 (KR) 10-2019-0159797
Mar. 26, 2020 (KR) 10-2020-0036845

(51) Int. Cl. G01R 33/34; G01R 33/36; A61B 5/055

(52) U.S. Cl. CPC G01R 33/34; G01R 33/3664; A61B 5/055

(57) ABSTRACT

Disclosed is a magnetic resonance imaging apparatus and a magnetic resonance imaging method using same, the magnetic resonance imaging apparatus comprising: a pair of end coils disposed at the top and bottom ends, respectively, having a ring shape, and having a shape in which several spaces cut out of a circumferential shape are connected by switching module; a plurality of leg coils connecting the pair of end coils; and the switching module respectively disposed between the pair of end coils and the plurality of leg coils, wherein the switching module include a high frequency transmission/reception coil opened by a first frequency and shorted by a second frequency different from the first frequency.

### TECHNICAL FIELD

The present disclosure relates to a radio frequency coil device for obtaining nuclear magnetic resonance signals of different nuclides without affecting a nuclear magnetic resonance (NMR) signal detected from an atomic nucleus, which is previously selected, within a magnetic resonance imaging system and a method for operating the same.

### BACKGROUND ART

A magnetic resonance imaging method is to acquire images of living tissues of the human body by using Nuclear Magnetic Resonance (NMR). According to the method, safety is ensured because radioactivity is not used, which is different from that of other imaging devices. In addition, the method is a non-invasive manner, so the higher-resolution image of the living tissue is acquired. Accordingly, the method has been widely utilized in medical fields. Recently, as the magnetic resonance imaging method is utilized for a functional MRI to analyze information related to a brain function, more various types of information may be acquired using the MRI.

Meanwhile, although a magnetic resonance image is obtained with respect to hydrogen mainly existing in a human body, a magnetic resonance image may be obtained from a different nuclide, and a signal may be detected from the different nuclide to observe the change in human metabolism and to diagnose diseases by acquiring the magnetic resonance signal from other nuclides (different nuclide; non-hydrogen). In this case, a radio frequency coil is additionally used to transmit/receive a signal by using a magnetic resonance frequency corresponding to the different nuclide.

A different nuclide utilized in a magnetic resonance image or a spectroscopy includes carbon (^{13 C}), sodium (²³Na), phosphorus (^{31 P}), and fluorine (¹⁹F). Among them, recently, the magnetic resonance images for carbon (¹³C) and sodium (²³Na) have been relatively actively studied.

In general, a hydrogen magnetic resonance image is obtained to exactly detect the location of an examination target before obtaining a magnetic resonance image signal of a different nuclide from the examination target. To this end, typically, a hydrogen radio frequency coil, which is commercially supplied, has been used.

Conventionally, to obtain the magnetic resonance signal of the different nuclide, a dual-tuned radio frequency coil, which operates at resonance frequencies of the two nuclides of hydrogen and the different nuclide, has been used to obtain both magnetic resonance signals of the two nuclides, instead of an existing hydrogen radio frequency coil. In this case, a coil structure and a coil switching process are complicated, and a signal to noise ratio (SNR) or the uniformity of an image may be degraded.

### DETAILED DESCRIPTION OF THE INVENTION

### Technical Problem

The present disclosure provides a magnetic resonance imaging system, in which a novel different nuclide (non-hydrogen) coil is provided inside an existing hydrogen radio frequency coil, and additionally a different nuclide radio frequency transmit/receive coil to exactly transmit/receive a different nuclide signal without affecting an electromagnetic wave of a hydrogen resonance frequency, such that images of the two nuclides are acquired without changing an MRI system employing an existing hydrogen coil, and a magnetic resonance imaging method using the same.

In addition, the present disclosure provides a magnetic resonance imaging system including a radio frequency transmit/receive coil unit to exactly detect a position for detecting a different nuclide signal by overlapping an existing magnetic resonance image of a hydrogen component, and a magnetic resonance imaging method.

The technical problems to be solved by the present disclosure are not limited to the aforementioned problems, and any other technical problems not mentioned herein will be clearly understood from the following description by those skilled in the art to which the present disclosure pertains.

### Technical Solution

According to an embodiment of the present disclosure, a radio frequency transmit/receive coil may include a pair of end coils provided at an upper end and a lower end of the radio frequency transmit/receive coil and having a ring shape; a plurality of leg coils to connect the pair of end coils to each other; and switching module disposed between the pair of end coils and the plurality of leg coils. The switching module may be open by a first frequency, and shorted by a second frequency different from the first frequency.

The end coil may include an upper coil disposed at the upper end and a lower coil disposed at the lower end. The upper coil and the lower coil include at least one spacing areas spaced in a circumference direction. The switching module may be disposed in the spacing area to connect the upper coil or the lower coil spaced.

In addition, the switching module may include a parallel-resonance circuit including an inductor and a first capacitor and a second capacitor which is series-connected to the parallel-resonance circuit.

In addition, the switching module may include a parallel-resonance circuit including a capacitor and a first inductor and a second inductor which is series-connected to the parallel-resonance circuit.

In addition, the first frequency may be a resonance frequency for exciting a hydrogen atom nucleus, and the second frequency may be a resonance frequency for exciting a non-hydrogen atom nucleus.

Meanwhile, according to an embodiment of the present disclosure, a magnetic resonance imaging (MRI) device may include a processor to determine pulse sequences applied to a target placed inside a static magnetic field, a first radio frequency transmit/receive coil unit which applies a first radio frequency pulse having a first frequency for exciting a first atomic nucleus included in the target and receives a first magnetic resonance signal emitted by the first radio frequency pulse; a second radio frequency transmit/receive coil unit to apply a second radio frequency pulse having a second frequency for exciting a second atomic nucleus included in the target and receive a second magnetic resonance signal emitted by the second radio frequency pulse; and a signal acquiring unit to process the first magnetic resonance signal and the second magnetic resonance signal. When the first radio frequency pulse is applied to the target through the first radio frequency transmit/receive coil unit, the operation of the second radio frequency transit/receive coil unit may be stopped.

In addition, the second radio frequency transmit/receive coil unit may include a switching module which is open by the first frequency, and shorted by the second frequency different from the first frequency.

The first radio frequency transmit/receive coil unit may include a first radio frequency transmit coil unit may include a first radio frequency transmit coil unit to apply a first radio frequency pulse having a first frequency for exciting a first atomic nucleus included in the target, and a first radio frequency receive coil unit to receive a first magnetic resonance signal emitted by the first radio frequency pulse. The first radio frequency receive coil unit is interposed between the second radio frequency transmit/receive coil unit and the target, shorted at the first frequency, and open at the second frequency.

In addition, a switching module of a second radio frequency transmit/receive coil unit may include a parallel-resonance circuit including an inductor and a first capacitor and a second capacitor which is series-connected to the parallel-resonance circuit.

In addition, the first frequency may be a resonance frequency for exciting a hydrogen atomic nucleus and the second frequency may be a resonance frequency for exciting a non-hydrogen atomic nucleus.

In addition, the second radio frequency transmit/receive coil unit may be interposed between the target and the first radio frequency transmit/receive coil unit.

According to an embodiment of the present disclosure, a magnetic resonance imaging (MRI) method may include (I) applying a first radio frequency pulse having a first frequency for exciting a first atomic nucleus included in a target placed in a static magnetic field, (II) receiving a first magnetic resonance signal emitted by the first radio frequency pulse applied to the first atomic nucleus, (III) applying a second radio frequency pulse having a second frequency for exciting a second atomic nucleus included in the target, (IV) receiving a second magnetic resonance signal emitted by the second radio frequency applied to the second atomic nucleus, and (V) generating an image of the target by using the first magnetic resonance signal and the second magnetic resonance signal.

In addition, the (I) step and the (II) step may be performed by the first radio frequency transmit/receive coil unit to apply the first radio frequency pulse having the first frequency for exciting the first atomic nucleus included in the target and to receive the first magnetic resonance signal emitted by the first frequency pulse, and the (III) step and the (IV) step may be performed by the second radio frequency transmit/receive coil unit to apply the second radio frequency pulse having the second frequency for exciting the second atomic nucleus included in the target and to receive the second magnetic resonance signal emitted by the second radio frequency pulse. When the first radio frequency pulse is applied to the target through the first radio frequency transmit/receive coil unit in the (I) step, the operation of the second radio frequency transmit coil unit may be stopped.

In addition, the (I) step may be performed by the first radio frequency transmit coil unit to apply the first radio frequency pulse having the first frequency for exciting the first atomic nucleus included in the target and the (II) step may be performed by the first radio frequency receive coil unit to receive the first magnetic resonance signal emitted by the first frequency pulse, The (III) step and (IV) step may be performed by the second radio frequency transmit/receive coil unit to apply the second radio frequency pulse having the second frequency for exciting the second atomic nucleus included in the target and to receive the second magnetic resonance signal emitted at the second radio frequency pulse. When the first radio frequency pulse is applied to the target through the first radio frequency transmit coil unit in the (I) step, the operation of the second radio frequency transmit/receive coil unit and the operation of the first radio frequency receive coil unit may be stopped. When the second radio frequency pulse is applied to the target through the second radio frequency transmit/receive coil unit in the (III) step, the operation of the first radio frequency receive coil unit may be stopped.

In addition, the second radio frequency receive coil unit may include a switching module, and the switching module of the second radio frequency transmit/receive coil unit is open by the first frequency in the (I) step, and shorted by the second frequency different from the first frequency in the (III) step.

In addition, the switching module of the second radio frequency transmit/receive coil unit may include a parallel-resonance circuit including an inductor and a first capacitor and a second capacitor which is series-connected to the parallel-resonance circuit.

In addition, the first radio frequency transmit/receive coil unit may include a switching module, and the switching module of the first radio frequency receive coil unit is shorted by the first frequency in the (I) step, and open by the second frequency different from the first frequency in the (III) step. In addition, a switching module may be further included and open when the first frequency is transmitted.

In addition, the switching module of the first radio frequency receive coil unit may include a parallel-resonance circuit including a capacitor and a first inductor and a second inductor which is series-connected to the parallel-resonance circuit.

In addition, in the step (I), the first frequency is a resonance frequency for exciting the hydrogen atomic nucleus, and the second frequency is a resonance frequency for exciting a non-hydrogen atomic nucleus in the step (III).

### Advantageous Effects of the Invention

According to an embodiment of the present disclosure, the novel different nuclide (non-hydrogen) coil is provided inside the existing hydrogen radio frequency coil, to exactly transmit/receive the different nuclide signal without affecting the electromagnetic wave of a hydrogen resonance frequency. Accordingly, a plurality of images are acquired without changing the MRI system employing the existing hydrogen coil.

According to an embodiment of the present disclosure, the novel different nuclide (non-hydrogen) coil is provided inside the existing hydrogen radio frequency coil, to exactly transmit/receive the different nuclide signal without affecting the electromagnetic wave of a hydrogen resonance frequency. Accordingly, a plurality of images are acquired without changing the MRI system employing the existing hydrogen coil.

In addition, according to an embodiment of the present disclosure, the position for detecting the different nuclide signal may be exactly detected in the overlap with the existing magnetic resonance image of the hydrogen component.

Meanwhile, the effects produced by the present disclosure are not limited to the aforementioned effects, and any other effects not mentioned herein will be clearly understood from the following description by those skilled in the art to which the present disclosure pertains.

### DESCRIPTION OF THE DRAWINGS

FIG. 1 is a view schematically illustrating the configuration of a magnetic resonance imaging (MRI) system, according to an embodiment of the present disclosure;

FIG. 2 is a view illustrating the detailed configuration of an MRI system, according to an embodiment of the present disclosure;

FIG. 3 is a view schematically illustrating a circuit configuration of a magnet device in a magnetic resonance imaging device, according to an embodiment of the present disclosure;

FIG. 4 is a circuit diagram schematically illustrating a circuit configuration of a first radio frequency transmit/receive coil unit in a magnetic resonance imaging device, according to an embodiment of the present disclosure;

FIG. 5 is a perspective view illustrating a radio frequency transmit/receive coil in a magnetic resonance imaging device, according to an embodiment of the present disclosure;

FIG. 6 is a plan view illustrating an unfolding state of a radio frequency transmit/receive coil of FIG. 5;

FIG. 7 is a circuit diagram illustrating the circuit configuration of a switching module (a circuit including a capacitor-inductor-capacitor configuration; CLC circuit) in a radio frequency transmit/receive coil, when a passing frequency is higher than an acquired frequency, according to an embodiment of the present disclosure;

FIG. 8 is a circuit diagram illustrating a parallel-resonance circuit in a CLC circuit;

FIG. 9 is a circuit diagram illustrating a series-resonance circuit in a CLC circuit;

FIG. 10 is a circuit diagram illustrating the circuit configuration of a switching module (a circuit including an inductor-capacitor-inductor configuration; LCL circuit) in a radio frequency transmit/receive coil, when a passing frequency is lower than an acquired frequency, according to an embodiment of the present disclosure;

FIG. 11 is a circuit diagram illustrating a parallel-resonance circuit in an LCL circuit;

FIG. 12 is a circuit diagram illustrating a series-resonance circuit in an LCL circuit;

FIG. 13 is a circuit diagram illustrating a groundbreaker in a radio frequency transmit/receive coil, according to an embodiment of the present disclosure;

FIG. 14 is a circuit diagram illustrating a 90-degree hybrid coupler in a radio frequency transmit/receive coil, according to an embodiment of the present disclosure;

FIG. 15 is a circuit diagram illustrating a transmit/receive switching circuit in a radio frequency transmit/receive coil, according to an embodiment of the present disclosure;

FIG. 16 is a circuit diagram illustrating that a one-channel radio frequency coil is connected to the transmit/receive switching circuit of FIG. 15;

FIGS. 17 and 18 illustrate a reflection attenuation constant as a function of a frequency through tuning and matching to be matched to a frequency of a signal, through a radio frequency transmit/receive coil, according to an embodiment of the present disclosure;

FIG. 19A is an image illustrating a magnetic resonance image of a swine heart captured using a hydrogen body coil, when a radio frequency transmit/receive coil is installed, according to an embodiment of the present disclosure;

FIG. 19B is an image illustrating a magnetic resonance image of a swine heart, when a radio frequency transmit/receive coil is not installed, according to an embodiment of the present disclosure;

FIG. 20A is a graph showing a 13C dynamic magnetic resonance spectroscopy showing the results of pyruvate metabolism obtained a total of 60 times every 3 seconds, according to an embodiment of the present disclosure;

FIG. 20B is a graph showing magnetic resonance spectroscopy signals of pyruvate, lactic acid, bicarbonate, and pyruvate hydrate over time, according to an embodiment of the present disclosure;

FIG. 20C is a graph illustrating the summed spectrum of the 13C dynamic spectrum acquired from the result of FIG. 20A;

FIG. 21A illustrates a pseudo-color map of an experimental result of free-induction decay chemical shift imaging (FID-CSI) and a pyruvate signal in a 4×4 spectrum grid of the swine heart area, according to an embodiment of the present disclosure;

FIG. 21B illustrates a 13C spectrum in the 4×4 spectrum grid, according to an embodiment of the present disclosure;

FIG. 21C is an image representing a pseudo-color map of a lactic acid signal in a swine heart, according to an embodiment of the present disclosure; and

FIG. 22 is a flowchart illustrating a magnetic resonance imaging method, according to an embodiment of the present disclosure.

### BEST MODE

Hereinafter, an embodiment of the present disclosure will be described in more detail with reference to the accompanying drawings. The embodiments of the present disclosure may be modified in various forms, and the scope of the present disclosure should not be construed to be limited by the embodiments of the present disclosure described in the following. The present embodiment is provided to describe the present disclosure for those skilled in the art more completely. Accordingly, the shapes and the like of the components in the drawings are exaggerated to emphasize clearer descriptions.

Hereinafter, the feature of the present disclosure will be described in detail with reference to exemplary embodiments and accompanying drawings to clarify solutions of problems to be solved according to the present disclosure. In the following description, the same reference numerals will be assigned to the same components even though the components are illustrated in different drawings. In addition, when the description is made with reference to a present drawing, a component in another drawing may be cited if necessary.

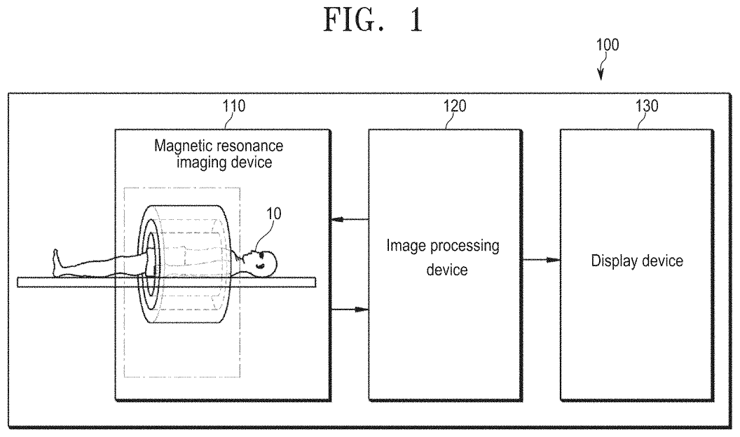

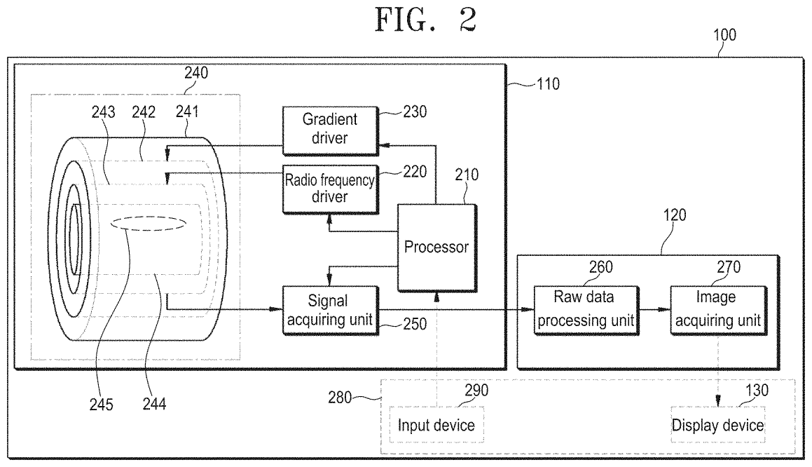

FIG. 1 is a view schematically illustrating the configuration of a magnetic resonance imaging (MRI) system, according to an embodiment of the present disclosure, and FIG. 2 is a view illustrating the detailed configuration of an MRI system, according to an embodiment of the present disclosure.

First, referring to FIG. 1, a magnetic resonance imaging system 100 includes a magnetic resonance imaging device 110, an image processing device 120, and a display device 130. In this case, devices constituting the magnetic resonance imaging system 100 may be included in one system in an integral type, which differs from the configuration illustrated in FIG. 1. Although FIG. 1 illustrates that the magnetic resonance imaging system 100 includes the display device 130, the present disclosure is not limited thereto. For example, the display device 130 may be provided outside the magnetic resonance imaging system 100.

In the magnetic resonance imaging system 100 illustrated in FIG. 1, only components related to the present embodiment are illustrated. Accordingly, it may be understood by those skilled in the art that general-purpose components are further included in addition to the components illustrated in FIG. 1.

The magnetic resonance imaging system 100 non-invasively acquires an image including information on a living tissue of a target using a magnetic field. In this case, the magnetic resonance imaging system 100 may be a hybrid magnetic resonance imaging (hybrid MRI) coupled to another medical imaging device such as a position emission tomography.

The magnetic resonance imaging device 110 applies a radio frequency magnetic field to the target 10, as the target 10 is positioned inside a static magnetic field.

The magnetic resonance imaging device 110 acquires a magnetic resonance signal emitted from the target 10 by the radio frequency magnetic field applied to the target 10, after the radio frequency magnetic field is applied. The magnetic resonance imaging device 110 outputs the acquired magnetic resonance signal to the image processing device 120.

The magnetic resonance imaging device 110 employs a magnetic resonance phenomenon of an atomic nucleus included in the target 10. The magnetic resonance phenomenon is a phenomenon in which atomic nuclei regularly aligned in the static magnetic field is applied with an electromagnetic wave having a specific frequency, is excited in a higher energy state, and then emits a weak electromagnetic wave while recovered to be in an original state. Atoms showing the magnetic resonance phenomenon ¹H, ³He, ¹⁹F, ²³Na, ³¹P, ¹³C, ¹²⁹Xe.

According to the present embodiment, the magnetic resonance imaging device 110 produces a magnetic resonance image by using magnetic resonance signals emitted from at least two atomic nuclei, which are in mutually different types, included in the target 10, instead of one type of an atomic nucleus.

The magnetic resonance imaging device 110 may apply, to the target 10, radio frequency pulses having frequencies in mutually different bands to excite mutually different types of atomic nuclei, such that the mutually different types of atomic nuclei are selectively excited, may apply a specific pulse sequence to each of the mutually different types of atomic nuclei, and may acquire magnetic resonance signals emitted by the radio frequency pulses applied to the mutually different types of atomic nuclei.

In this case, according to the present embodiment, the magnetic resonance imaging device 110 may acquire a magnetic resonance signal for a hydrogen atomic nucleus and a magnetic resonance signal for a non-hydrogen atomic nucleus, by using an existing hydrogen radio frequency coil without change and by providing a non-hydrogen radio frequency coil between the target 10 and the hydrogen radio frequency coil.

Meanwhile, the non-hydrogen radio frequency coil may include a switching module, and the switching module may be open at a magnetic resonance frequency of hydrogen and shorted at a magnetic resonance frequency of a non-hydrogen atomic nucleus.

Accordingly, the non-hydrogen atomic nucleus may acquire a magnetic resonance signal from other nuclides of a non-hydrogen, without affecting detecting a magnetic resonance signal of a hydrogen atom.

Meanwhile, the image processing device 120 generates a magnetic resonance image of the target 10 by using the magnetic resonance signal received from the magnetic resonance imaging device 110.

According to the present embodiment, the magnetic resonance imaging device 110 generates the magnetic resonance image, based on magnetic resonance signals acquired by using mutually different types of atomic nuclei, instead of one type of atomic nucleus. Accordingly, the magnetic resonance imaging device 110 may simultaneously acquire metabolic information as well as anatomical information of a biological subject.

In addition, the magnetic resonance imaging device 110 may acquire multiple parameters of metabolic information by using the magnetic resonance image acquired from the multiple types of atomic nuclei, thereby diagnosing diseases, such as lesions or tumors, which may be diagnosed using specific elements.

The display device 130 displays an image representing a living tissue of the target 10 by receiving the magnetic resonance image from the image processing device 120.

In addition, components illustrated in FIG. 1, the magnetic resonance imaging system 100 may further include a user interface to receive, from a user, various control parameters used to acquire the magnetic resonance signal by the magnetic resonance imaging device 110, and a memory to store the magnetic resonance image generated by the image processing device 120.

In addition, referring to FIG. 2, the magnetic resonance imaging system 100 includes the magnetic resonance imaging device 110, the image processing device 120, and a user interface unit 280. The magnetic resonance imaging device 110 includes a processor 210, a radio frequency driver 220, a gradient driver 230, a magnet device 240, and a signal acquiring unit 250. The magnet device 240 includes a main magnetic field coil unit 241, a gradient coil unit 242, a first radio frequency transmit/receive coil unit 243, and a second radio frequency transmit/receive coil unit 244.

In this case, when the first radio frequency transmit/receive coil unit 243 is used to transmit a first radio frequency, the first radio frequency transmit/receive coil unit 243 may be interposed between the second radio frequency transmit/receive coil unit 244 and the target 10, and may further include a first radio frequency receive coil unit 245 to receive a first magnetic resonance signal emitted by the first radio frequency pulse.

The image processing device 120 includes a raw data processing unit 260 and an image acquiring unit 270. The user interface unit 280 includes an input device 290 and the display device 130. A magnetic resonance imaging system 100 illustrated in FIG. 2 corresponds to one example of the magnetic resonance imaging system 100 illustrated in FIG. 1. Accordingly, the description of the magnetic resonance imaging system 100 of FIG. 1 is applicable to the description of the magnetic resonance imaging system 100 of FIG. 2. To this regard, the duplication thereof will be omitted.

The magnetic resonance imaging system 100 non-invasively acquires an image including information on a living tissue of a target using a magnetic field. In this case, the image may be a two dimensional image or a three dimensional image depending on a pulse sequence.

The magnetic resonance imaging device 110 applies a radio frequency pulse and a specific pulse sequence to the target 10 and acquires magnetic resonance signals emitted from the target 10, as the target 10 is positioned inside a magnetic field.

In the magnetic resonance imaging device 110, the processor 210 applies a radio frequency pulse and a pulse sequence to the target 10 and controls the overall magnetic resonance imaging operations of acquiring magnetic resonance signals. In addition, the processor 210 applies control signals to the radio frequency driver 220, the gradient driver 230, the magnet device 240, and the signal acquiring unit 250 of the magnetic resonance imaging device 110, and controls all components of the magnetic resonance imaging device 110 in response to the applied control signal.

The magnet device 240 may apply a static magnetic field, radio frequency pulses, and gradient signals to the target 10 and may acquire the magnetic resonance signals from the target 10, to acquire the magnetic resonance image for the living tissue of the target 10. The magnet device 240 includes the main magnetic field coil unit 241, the gradient coil unit 242, the first radio frequency transmit/receive coil unit 243, the second radio frequency transmit/receive coil unit 244, and may include the first radio frequency receive coil unit 245 including at least one first radio frequency receive coil if necessary. Meanwhile, the coil shapes of coil units 241, 242, 243, 244, and 245 included in the magnet deice 240 illustrated in FIG. 2 are not limited to the shape of FIG. 2. For example, the coil units 241, 242, 243, 244, and 245 may be realized in various shapes.

The main magnetic field coil unit 241 regularly aligns a plurality of atomic nuclei included in the target 10 by generating a static magnetic field. The plurality of atomic nuclei are aligned in a direction parallel to or opposite to a direction of the magnetic field, by the magnetic field corresponding to force applied to the atomic nuclei from the outside.

The gradient coil unit 242 applies a specific pulse sequence to each of mutually different types of the atomic nuclei. The gradient coil unit 242 applies, to the target 10, gradient signals, such as a selection gradient, a phase encoding gradient, and a frequency encoding gradient, for space encoding.

The gradient coil unit 242 may apply three types of gradients in x, y, and z-axis directions of the target 10. For example, the gradient coil unit 242 may obtain a transverse tomography image of the target 10 by applying gradient signals in the following manner. The gradient coil unit 242 applies the selection gradient to the region of interest (ROI) of the target 10 to acquire a tomographic image in the z-axis direction which is a longitudinal direction. The gradient coil unit 242 applies a frequency encoding gradient in the x-axis direction, and a phase encoding gradient in the y-axis direction, on a two-dimensional plane selective excited through the selection gradient and by a radio frequency for exciting the selected space. Accordingly, the magnetic resonance imaging system 100 may perform space encoding for the two-dimensional plane and may acquire a magnetic resonance image in two dimensions.

For another example, after applying a radio frequency pulse to excite a three-dimensional whole space without applying the selection gradient, the gradient coil unit 242 may apply a phase encoding gradient in the z-axis direction, in addition to a phase encoding gradient in the y-axis direction. Accordingly, the magnetic resonance imaging system 100 may perform space encoding for the three-dimensional plane and may acquire a magnetic resonance image in three-dimensions.

The gradient coil unit 242 may apply various types of pulse sequences to the target 10, in addition to examples described above. Although the above description has been made in that the gradient coil unit 242 applies the selection gradient in the z-axis direction, the present disclosure is not limited thereto. For example, the gradient coil unit 242 may perform two-dimensional selection gradient or three-dimensional space encoding by applying the selection gradient to the target 10, which is positioned in the static magnetic field, in a specific axis direction.

The gradient coil unit 242 may perform two-dimensional or three-dimensional space encoding by selectively applying a specific pulse sequence to each of at least two mutually different types of atomic nuclei.

The first radio frequency transmit/receive coil unit 243 applies, to the target 10, first frequency pulses having a first frequency in a preset band to excite the first atomic nucleus included in the target 10. In this case, the first atomic nucleus may be excited by the first frequency, which is preset, based on an intrinsic gyromagnetic ratio.

In this case, the first frequency for exciting the first atomic nucleus may be determined the intensity (Bₒ) of a magnetic field applied to the main magnetic field coil unit 241 and the intrinsic gyromagnetic ratio (γ) of the first atomic nucleus. The frequency for exciting atomic nuclei included in the target 10 is referred to as a procession frequency or a Larmor frequency.

In this case, the Larmor frequency (or f₀ [Hz) may be defined as in Equation 1.

ω₀=2πf₀=γB₀ Equation 1

In this case, γ denotes gyromagnetic ratio [rad/sec/T], and Bₒ denotes the intensity of an external magnetic field.

According to an embodiment, the first radio frequency transmit/receive coil unit 243 may apply, to the target 10, the first radio frequency pulse having the first frequency for exciting a hydrogen atomic nucleus.

In other words, as the first atomic nucleus is determined as hydrogen, the main magnetic field coil unit 241, the gradient coil unit 242, and the first radio frequency transmit/receive coil unit 243, which are used in an existing magnetic resonance imaging system, may be used without change.

Meanwhile, in a 1.0 Tesla magnetic resonance device, the gyromagnetic ratio of hydrogen used is 42.58 MHz/T, and an external magnetic field strength is 3.0 T in the magnetic resonance imaging system 100. Accordingly, the magnetic resonance frequency of hydrogen may be calculated as 127.74 MHz.

In addition, the first radio frequency transmit/receive coil unit 243 receives a magnetic resonance signal emitted by radio frequency pulses applied to the first atomic nucleus. The first radio frequency transmit/receive coil unit 243 acquires electromagnetic waves emitted when the atomic nucleus excited by the applied radio frequency pulse is recovered to be in the original state. In this case, the acquired electromagnetic wave corresponds to a magnetic resonance signal.

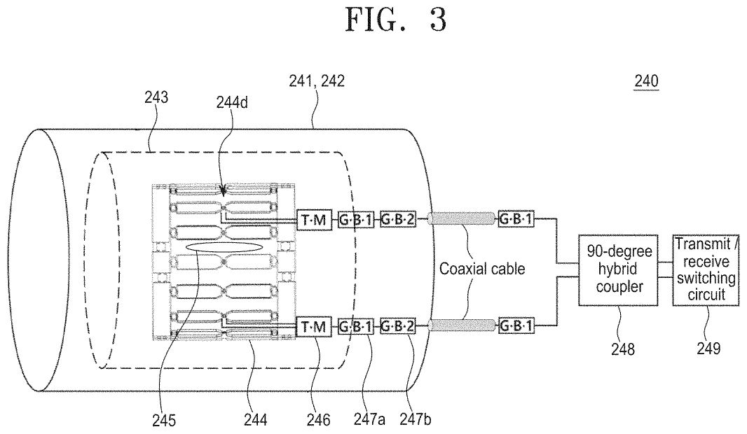

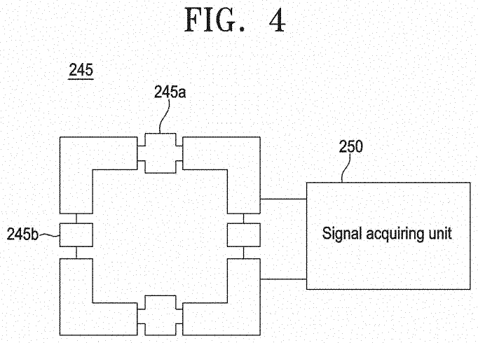

FIG. 3 is a view schematically illustrating a circuit configuration of a magnet device in a magnetic resonance imaging device 110, according to an embodiment of the present disclosure, and FIG. 4 is a circuit diagram schematically illustrating a circuit configuration of a first radio frequency receive coil unit 245 additionally provided for receiving, when the first radio frequency transmit/receive coil unit 245 is used for transmission in a magnetic resonance imaging device 110, according to an embodiment of the present disclosure.

Meanwhile, referring to FIGS. 2 to 4, in the magnetic resonance imaging device 110, the first radio frequency transmit/receive coil unit 243 performs only a transmission function of applying a first radio frequency pulse having a first frequency for exciting a hydrogen atomic nucleus, instead of performing both transmission and reception functions of the first frequency, and the first radio frequency receive coil unit 245 may be additionally provided to receive the magnetic resonance signal emitted by radio frequency pulses applied to the first atomic nucleus.

The second radio frequency transmit/receive coil unit 244 may be disposed inside the first radio frequency transmit/receive coil unit 243. In other words, the second radio frequency transmit/receive coil unit 244 may be interposed between the first radio frequency transmit/receive coil unit 243 and the target 10.

The second radio frequency transmit/receive coil unit 244 applies, to the target 10, second frequency pulses having a second frequency in a preset band to excite the second atomic nucleus included in the target 10. In this case, the second atomic nucleus may include an atomic nucleus different from the first atomic nucleus.

In this case, the second frequency for exciting the second atomic nucleus may be determined by the intensity (Bₒ) of a magnetic field applied to the main magnetic field coil 241 and the intrinsic gyromagnetic ratio (γ) of the second atomic nucleus.

Meanwhile, the second radio frequency transmit/receive coil unit 244 may include a switch member which is open at the first frequency, and shorted at the second frequency different from the first frequency.

In this case, the switching module may be described below with reference to FIG. 3.

According to an embodiment, the second radio frequency transmit/receive coil unit 244 may apply, to the target 10, the second radio frequency pulse having the second frequency for exciting the atomic nucleus of carbon-13.

Meanwhile, in a 1.0 Tesla magnetic resonance device, the gyromagnetic ratio of carbon-13 used is 10.71 MHz/T, and the intensity of an external magnetic field is 3.0 T in the magnetic resonance imaging system 100 according to an embodiment of the present disclosure. Accordingly, the magnetic resonance frequency of carbon-13 may be calculated as 32.13 MHz.

In addition, the second radio frequency transmit/receive coil unit 244 receives a magnetic resonance signal emitted by radio frequency pulses applied to the second atomic nucleus. The second radio frequency transmit/receive coil unit 244 acquires electromagnetic waves discharged when the atomic nucleus excited by the applied radio frequency pulse is recovered to be in the original state. In this case, the acquired electromagnetic wave corresponds to a magnetic resonance signal from the second atomic nucleus.

The signal acquiring unit 250 performs signal processing by acquiring magnetic resonance signals output from the first radio frequency transmit/receive coil unit 243 and the second radio frequency transmit/receive coil unit 244, or the first radio frequency receive coil unit 245 and the second radio frequency transmit/receive coil unit 244. For example, magnetic resonance signals received from the first radio frequency transmit/receive coil unit 243 and the second radio frequency transmit/receive coil unit 244 have significantly weak intensities, and the signal acquiring unit 250 may amplify the first radio frequency transmit/receive coil unit 243 and the second radio frequency transmit/receive coil unit 244 by using an amplifier. In addition, the signal acquiring unit 250 may demodulate the magnetic resonance signals by using a demodulator, or may convert the magnetic resonance signals into digital signals by using an analog to digital converter (ADC).

As described above, the signal acquiring unit 250 may split the received magnetic resonance signals into magnetic resonance signals, which correspond to mutually different types of atomic nuclei, depending on relevant frequency bands, by using a filter. However, the present disclosure is not limited thereto. The signal acquiring unit 250 may process various signals with respect to the magnetic resonance signals acquired by the first radio frequency transmit/receive coil unit 243 and the second radio frequency transmit/receive coil unit 244.

The magnetic resonance signals output from the magnetic resonance imaging device 110 correspond to raw data, and image processing is required to generate an image of the cell tissue of the target 10. Accordingly, the image processing device 120 performs image processing to generate images for magnetic resonance signals output from the magnetic resonance imaging device 110. The image processing device 120 includes the raw data processing unit 260 and the image acquiring unit 270.

The raw data processing unit 260 forms a k-space including the information on a position by using magnetic resonance signals output from the magnetic resonance imaging device 110.

The image acquiring unit 270 generates an image of the target 10 by using image data processed by the raw data processing unit 260.

In detail, the image acquiring unit 270 may receive k-space data constituting a k-space from the raw data processing unit 260, may perform Fourier Transform for the k-space data, and may acquire a magnetic resonance image for the living tissue of the target 10.

The user interface unit 280 acquires input information from a user and displays output information. Although the input device 290 and the display device 130 are separated from each other as in FIG. 2, for the convenience of explanation, the present disclosure is not limited thereto. For example, the input device 290 and the display device 130 may be unified in one device and operated.

The input device 290 may receive, from a user, at least two types of atomic nuclei, which are to be used in magnetic resonance imagining, of multiple types of atomic nuclei included in the target 10, as input information. The input device 290 may receive, as input information, various control parameters for determining the shape of a specific pulse sequence applied to the target 10 through each of the gradient coil unit 242, the first radio frequency transmit/receive coil unit 243, and the second radio frequency transmit/receive coil unit 244. Alternatively, the input device 290 may receive, as input information, a region of interest (ROI), which is to be used to acquire a magnetic resonance image, of the target 10. However, the present disclosure is not limited thereto. For example, the input device 290 may receive various pieces of information as input information. For example, the input device 290 may include a device, such as a keyboard or a mouse, provided in the magnetic resonance imaging system 100 and a software module to drive the components.

The display device 130 displays a target image generated by the image acquisition unit 270. For example, the display device 130 may include a device, such as a display panel or a monitor, provided in the magnetic resonance imaging system 100 and a software module to drive the components.

Although FIG. 2 illustrates that the magnetic resonance imaging system 100 includes the display device 130, the present disclosure is not limited thereto. For example, the display device 130 may be provided outside the magnetic resonance imaging system 100.

According to the magnetic resonance imaging system 100 of the present embodiment, the magnetic resonance image is acquired by sequentially exciting at least two atomic nuclei of the multiple types of atomic nuclei included in the target 10, thereby acquiring the anatomical information and metabolic information without matching different types of individual images, such as a PET-MRI image.

Accordingly, the time and the effort required to merge individual images acquired from atomic nuclei into each other may be reduced, and spatial and temporal errors of the individual images and the errors caused in the merging process may be reduced, such that the image is exactly acquired.

In addition, when a specific is tracked using specific elements or tissue activities such as the migration or proliferation of the tissue is observed, the structure information of the tissue and metabolic information on the tissue are simultaneously acquired. Accordingly, the position of the tissue may be exactly acquired.

The atomic nuclei of ³He, ¹³C, ¹²⁹Xe corresponding to hyperpolarized gas and the atomic nucleus of ¹H are excited to acquire magnetic resonance signals. Accordingly, it may be understood through one target that the information on the distribution and the relative positions of atomic nuclei, as an image of gas exchange inside a lung is acquired from a hyperpolarized atomic nucleus and an image of the lung tissue structure is acquired from the hydrogen atomic nucleus.

As described above, the multiple types of atomic nuclei are excited with respect to one target to acquire the structural information and metabolic information of a biological subject. Accordingly, the tissue may be observed and traced in real time. Further, diseases such as lesions or tumors may be exactly diagnosed.

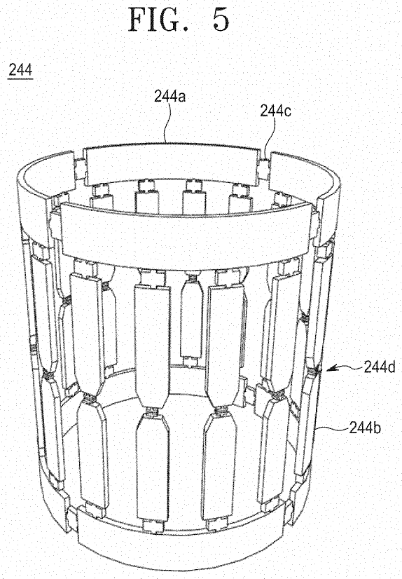

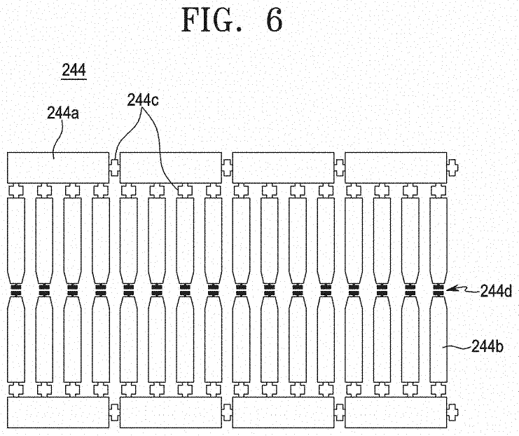

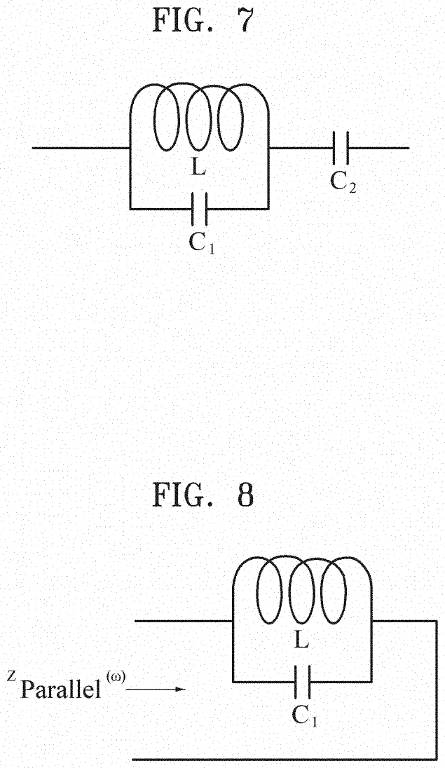

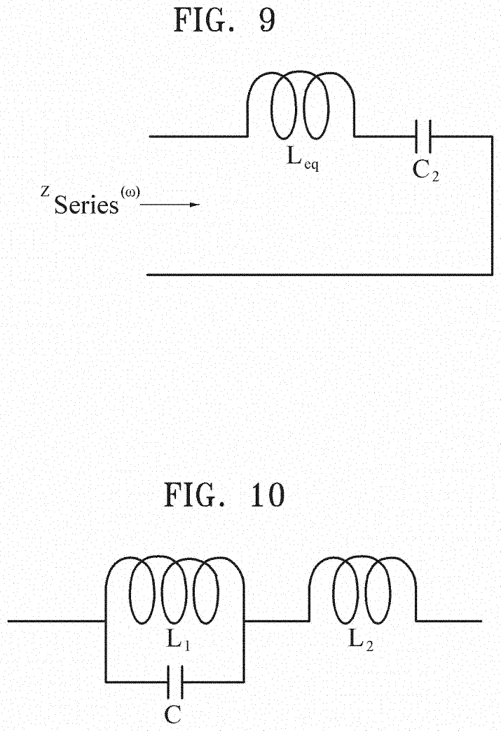

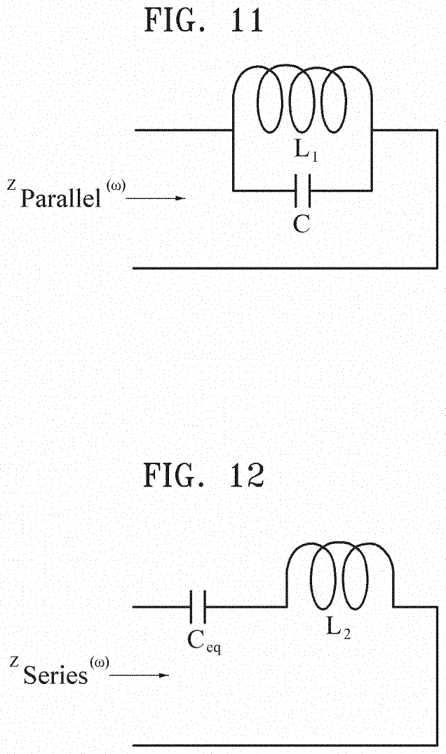

FIG. 5 is a perspective view illustrating a radio frequency transmit/receive coil in the magnetic resonance imaging device 110 according to an embodiment of the present disclosure, FIG. 6 is a plan view illustrating an unfolding state of a radio frequency transmit/receive coil of FIG. 5, FIG. 7 is a circuit diagram illustrating the circuit configuration of a switching module in a radio frequency transmit/receive coil according to an embodiment of the present disclosure, FIG. 8 is a circuit diagram illustrating a parallel-resonance circuit, and FIG. 9 is a circuit diagram illustrating a series-resonance circuit.

First, referring to FIG. 3, the magnet device 240 may include the main magnetic field coil unit 241, the gradient coil unit 242, the first radio frequency transmit/receive coil unit 243, the second radio frequency transmit/receive coil unit 244, the first radio frequency receive coil unit 245, a tuning and matching circuit 246, ground breakers 247a and 247b, a 90-degree hybrid coupler 248, and a transmit/receive switching circuit 249.

Referring to FIGS. 5 and 6, the second radio frequency transmit/receive coil unit 244 may include a pair of end coils 244a, a leg coil 244b, a switching module 244c, and a capacitor 244d.

In other words, the second radio frequency transmit/receive coil unit 244 may be formed in a cage-type coil shape.

Meanwhile, although not illustrated, the pair of end coils 244a, the leg coil 244b, the switching module 244c, and the capacitor 244d may be disposed on an outer circumferential surface of a cylindrical housing (not illustrated).

The pair of end coils 244a may include copper conductors, may be disposed at upper and lower ends, respectively, and may have a ring shape.

The leg coil 244b may include a copper conductor, and may be formed to connect the pair of end coils 244a. A plurality of leg coils 244b may be provided.

The switching module 244c may be interposed among the pair of end coils 244a and the plurality of leg coils 244b, respectively. Here, the switching module 244c may be open by a first frequency and shorted by a second frequency different from the first frequency.

Meanwhile, each of the upper coil 244a and the lower coil 244a may include at least one spacing area spaced in the circumferential direction, and the switching modules 244c may be disposed in each spacing area in the upper coil 244a, in the lower coil 244a, and between the plurality of leg coils 244b.

Meanwhile, the switching module 244c is schematically illustrated in FIGS. 3 to 6. When a first passing frequency is greater than a second acquired frequency, the switching module 244c may include a parallel-resonance circuit including an inductor ‘L’ and a first capacitor ‘C₁’, and a passive element including a second capacitor ‘C₂’ series-connected to the parallel-resonance circuit (capacitor-inductor-capacitor resonance circuit; CLC). When the first passing frequency is lower than the second acquired frequency, the switching module 244c may include a parallel-resonance circuit including a first inductor ‘L₁’ and a capacitor ‘C’, and a passive element including a second inductor ‘L₂’ series-connected to the parallel-resonance circuit (an inductor-capacitor-inductor (LCL) resonance circuit).

When the first passing frequency is greater than the second acquired frequency, that is, in the CLC resonance circuit, an open switch employing only the passive element may include a parallel-resonance circuit (LC circuit) as illustrated in FIG. 8, may be open at a first resonance frequency (ω) which is preset. In this case, the impedance (Zₚₐᵣₐₗₗₑₗ) in the parallel LC circuit may be calculated as illustrated in Equation 2.

In this case, the first resonance frequency (ω) preset may be a resonance frequency of a hydrogen atomic nucleus.

$${Z}_{parallel} ( ω ) = {jL}_{eq} ω = jL {ω ( 1 - \frac{ω}{{ω}_{eff}} )}^{- 1} \qquad Equation 2$$

$${ω}_{off} = \frac{1}{\sqrt{{LC}_{1}}} , {L}_{eq} = {L ( 1 - \frac{ω}{{ω}_{off}} )}^{- 1}$$

In this case, when ω=ω_{off}, a parallel-resonance state is formed, such that Zₚₐᵣₐₗₗₑₗ(ω)=∞(infinite), that is, becomes in an open state. When ω<ω_{off}, a value in a bracket becomes positive, so the Zₚₐᵣₐₗₗₑₗ(ω) totally functions as an inductor. Accordingly, at this frequency, a series-resonance is made with an additional capacitor (second capacitor), the whole circuit may be shorted.

In addition, a short switch employing only the passive element may include a series-LC circuit as illustrated in FIG. 9, and may be shorted at a second resonance frequency (ω). In this case, the impedance (Zₛₑᵣᵢₑₛ) of the series LC circuit may be calculated as illustrated in following Equation 3.

In this case, the set second resonance frequency (ω) may be a resonance frequency of a carbon-13 atomic nucleus.

$${Z}_{series} ( ω ) = {jL}_{eq} ω ( 1 - \frac{{ω}_{on}^{2}}{ω} ) \qquad Equation 3$$

$${ω}_{on} = \frac{1}{\sqrt{{L}_{eq} {C}_{2}}}$$

In this case, the parallel-connection circuit (L-C₁) operates as having an inductance L_{eq}, that is, operates as one inductor, and has an equivalence inductor of the parallel-resonance circuit as illustrated in FIG. 8.

In other words, when a frequency for shorting is ωₒₙ, and a frequency for opening is ω_{off} in the circuit of the switching module 244c, and when ωₒₙ<ω_{off}, the switching module 244c operates as an open/short switching circuit as illustrated in FIGS. 6 to 7 by combining the two resonance circuits of a parallel-resonance circuit and a series-resonance circuit.

According to an embodiment, in detail, the second radio frequency transmit/receive coil unit 244 may be defined as a 13-carbon cage coil in a low pass filter type, and a switching module 244c of the second radio frequency transmit/receive coil unit 244 may operate as an open circuit at 127.74 MHz which is a resonance frequency of a hydrogen. The switching module 244c may be shorted at 32.13 MHz which the 13-carbon resonance frequency.

In other words, since the second radio frequency transmit/receive coil unit 244 operates as a cage coil in a low pass filter type for transmitting or receiving a carbon magnetic resonance circuit, ωₒₙ and ω_{off} are a 13-carbon magnetic resonance frequency and a hydrogen magnetic resonance frequency, respectively. Among them, the inductor ‘L’ and capacitors ‘C₁’ and ‘C₂’ of the switching module 244c constituting the frequency switching circuit may be calculated as in following Equation 4.

$${C}_{1} = \frac{1}{L {ω}_{off}^{2}} \qquad Equation 4$$

$${C}_{2} = \frac{{ω}_{off}^{2} - {ω}_{on}^{2}}{L {ω}_{on}^{2} {ω}_{off}^{2}}$$

$$\frac{{ω}_{off}}{{ω}_{on}} = \sqrt{\frac{1 + {C}_{2}}{{C}_{1}}}$$

In this case, as described above, when the intensity of an external magnetic field is 3.0 T, a hydrogen resonance frequency and a carbon resonance frequency are 127.74 MHz and 32.13 MHz, respectively. Accordingly, values of the inductor ‘L’ and the capacitors ‘C₁’ and ‘C₂’ may be calculated as L=277 nH, C₁=5.6 pF, and C₂=83 pF.

In this case, although ‘L’, ‘C₁’, and ‘C₂’ may be employed in the various combinations thereof to satisfy two resonance conditions, that is, a parallel resonance condition of ‘L’ and ‘C₁’ and a series-resonance condition of a parallel-connection circuit (L-C₁) expressed with an equivalent inductor and ‘C₂’, it is preferred that ‘L’, ‘C₁’, and ‘C₂’ are selected as smaller values to increase the value of ‘Q’ of capacitors and to decrease the size of the inductor. In this case, an excessively small value may be easily affected through the coupling with a peripheral environment, so the capacitors ‘C₁’ and ‘C₂’ are preferably set in the range of 5 pF to 100 pF, and an inductor ‘L’ is preferably set as a value of several hundred nH.

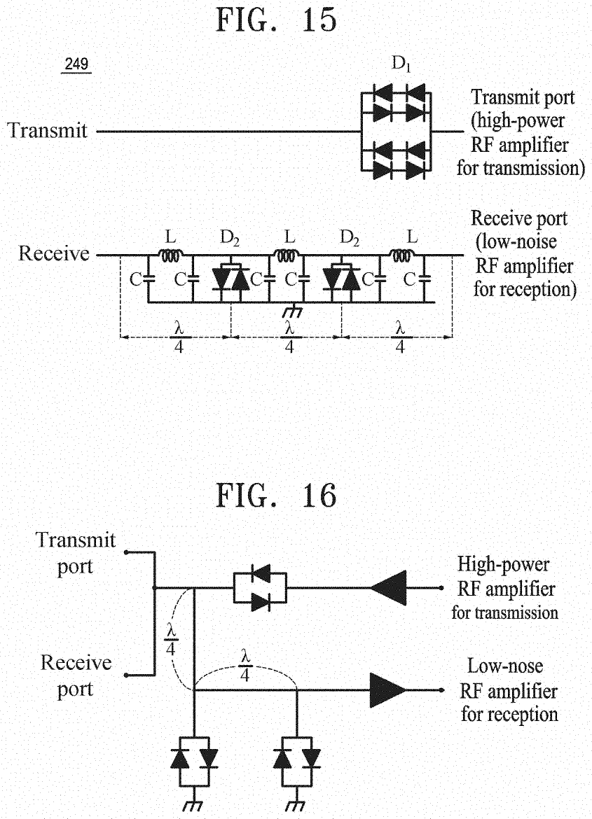

Meanwhile, in a third coil installed in a second different nuclide coil to acquire a hydrogen signal, an open switch may include the parallel-resonance circuit (LC circuit) employing only the passive element as illustrated in FIG. 11, and may open at the set passing frequency (ω) in the case of the switching module LCL, that is, when the passing frequency is lower than the acquired frequency, that is, in the case of the resonance circuit LCL. In this case, the impedance ‘Zₚₐᵣₐₗₗₑₗ’ of the parallel LC circuit may be calculated as in the following Equation 5.

In this case, the set passing frequency (ω) may be a resonance frequency of an atomic nucleus in a different nuclide.

$${Z}_{parallel} ( ω ) = \frac{1}{jC ω} {( 1 - \frac{{ω}_{off}^{2}}{{ω}^{2}} )}^{- 1} \qquad Equation 5$$

In this case, when ω=ω_{off}, a parallel-resonance state is formed, such that Zₚₐᵣₐₗₗₑₗ(ω)=∞(infinite), that is, becomes in an open state. When ω>ω_{off}, a value in a bracket becomes positive, so the Zₚₐᵣₐₗₗₑₗ(ω) totally functions as a capacitor. Accordingly, at this frequency, a series-resonance is made with an additional inductor (second inductor), the whole circuit may be shorted.

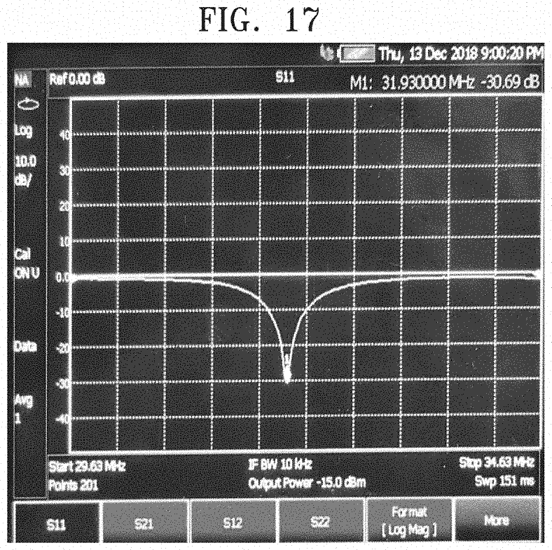

In addition, a short switch employing only the passive element may include a series-LC circuit as illustrated in FIG. 12, and may be shorted at a set acquired frequency (ω). In this case, the impedance (Zₛₑᵣᵢₑₛ) of the series LC circuit may be calculated as illustrated in following Equation 6.

In this case, the set acquired frequency (ω) may be a resonance frequency of a hydrogen atomic nucleus.

$${Z}_{series} ( ω ) = \frac{1}{{jC}_{eq} ω} ( 1 - \frac{{ω}_{on}^{2}}{{ω}^{2}} ) \qquad Equation 6$$

In this case, the parallel-connection circuit (L1-C) operates as having an inductance C_{eq}, that is, operates as one capacitor, and has an equivalence capacitor of the parallel-resonance circuit as illustrated in FIG. 11.

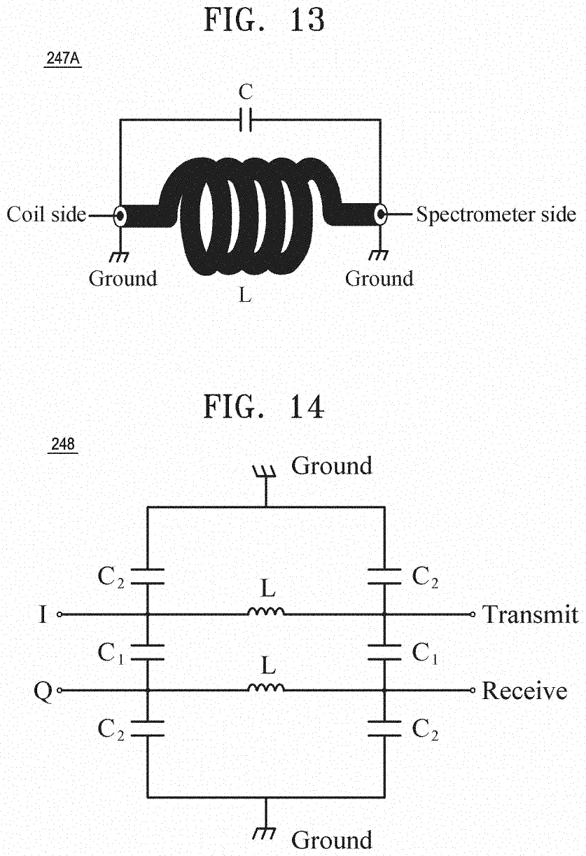

In other words, when a frequency for shorting is ωₒₙ, and a frequency for opening is ω_{off} in the circuit of the switching module 244c, and when ωₒₙ>ω_{off}, the switching module 244c operates as an open/short switching circuit as illustrated in FIGS. 4 to 10 by combining the two resonance circuits of a parallel-resonance circuit and a series-resonance circuit.

In detail, according to an embodiment, the first radio frequency receive coil unit 245 may be defined as a hydrogen coil having a ring-shape loop structure and may include a switching module 245a and a capacitor 245b. The first radio frequency receive coil, which has a ring shape including the switching module, includes at least one switching module spaced apart from each other in a circumference direction. The switching module 245a of the first radio frequency receive coil unit 245 operates as an open circuit at the 13-carbon resonance frequency of 32.13 MHz, and may operate as a shorted circuit at the hydrogen resonance frequency of 127.74 MHz.

In other words, since the first radio frequency transmit/receive coil unit 245 operates as a loop coil to receive a hydrogen magnetic resonance signal, ωₒₙ and ω_{off} are magnetic resonance frequencies of a hydrogen and carbon-13 in the 3.0 T magnetic resonance device. Among them, in the switching module 245a including the switching circuit, the values of the inductors ‘L₁’ and ‘L₂’ and a capacitor ‘C’ may be calculated as in Equation 7.

$${L}_{1} = \frac{1}{C {ω}_{on}^{2}} \qquad Equation 7$$

$${L}_{2} = \frac{{ω}_{on}^{2} - {ω}_{off}^{2}}{C {ω}_{on}^{2} {ω}_{off}^{2}}$$

$$\frac{{ω}_{on}}{{ω}_{off}} = \sqrt{\frac{1 + {L}_{2}}{{L}_{1}}}$$

In this case, as described above, when the intensity of the external magnetic field is 3.0 T, the resonance frequencies of hydrogen and carbon are 127.74 MHz and 32.13 MHz, respectively. Accordingly, the device values of the inductors ‘L₁’ and ‘L₂’ and the capacitor ‘C’ may be calculated as L₁=0.06354 pH, L₂=0.4291 pH, C=0.015 pF.

In this case, although ‘C’, ‘L₁’, and ‘L₂’ may be employed in the various combinations thereof to satisfy two resonance conditions, that is, a parallel resonance condition of ‘L₁’ and ‘C’ and a series-resonance condition of a parallel-connection circuit (L₁-C) expressed with an equivalent capacitor and ‘L₂’, it is preferred that ‘L’, ‘C₁’, and ‘C₂’ are selected as smaller values, as much as possible to increase the value of ‘Q’ of capacitors and to decrease the size of the inductor.

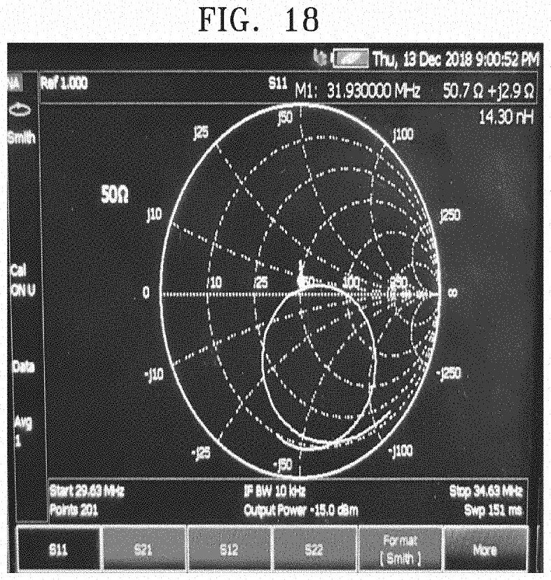

FIG. 13 is a circuit diagram illustrating a groundbreaker in a radio frequency transmit/receive coil, according to an embodiment of the present disclosure.

Meanwhile, to prevent the flow of a current having a hydrogen resonance frequency or a different nuclide resonance frequency, which causes noise or heat from a coaxial cable transmitting or receiving a different nuclide resonance signal, a balun or a groundbreaker may be disposed.

In this case, since the LC parallel-resonance frequency is a hydrogen or non-hydrogen (different nuclide) magnetic resonance frequency. According to the present disclosure, since both the non-hydrogen magnetic resonance frequency and the hydrogen magnetic resonance frequency are employed simultaneously or sequentially, when both groundbreakers 247a and 247b for two magnetic resonance frequencies are employed to be installed in coil signal lines for the different nuclide resonance frequency, the nose or the heat may be prevented. In this case, regarding the structures of the groundbreakers 247a and 247b, various types of groundbreakers may be employed.

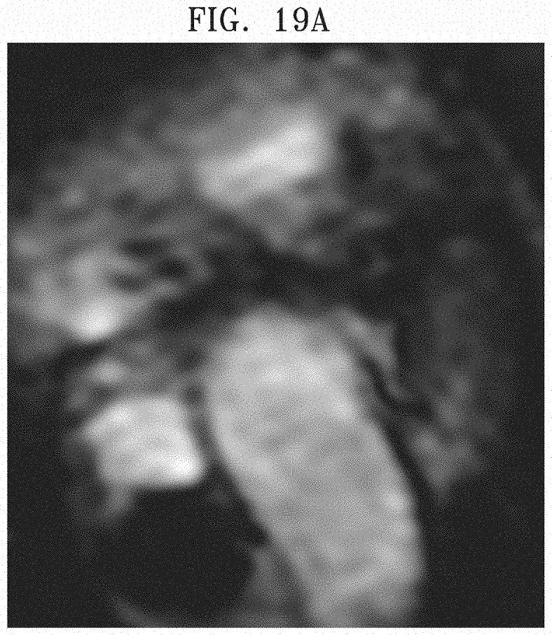

FIG. 14 is a circuit diagram illustrating the 90-degree hybrid coupler 248 in a radio frequency transmit/receive coil, according to an embodiment of the present disclosure.

Meanwhile, referring to FIG. 14, the 90-degree hybrid coupler 248 is a device including four ports and transmits a signal, which is input from a transmit port, to the second radio frequency transmit/receive coil unit through two ports ‘I’ and ‘Q’ by making a phase difference of 90 degrees and attenuating at least 3 dB.

The signals input into the two ports ‘I’ and ‘Q’ at the side of the second radio frequency transmit/receive coil 244 are attenuated by 3 dB, and combined to have the phase difference of −90 degrees. Accordingly, the signals are increased up to 3 dB and output through receive ports. In this case, the attenuation of 3 dB refers to that power is attenuated to a half of input power, a voltage is attenuated to 1\sqrt{2}times of an input voltage.

The 90-degree hybrid coupler 248 is designed and manufactured such that a circuit is linearly symmetrical. When a switching circuit is absent, the 90-degree hybrid coupler 248 is symmetrical without distinguishing between input/output ports. Values in the 90-degree hybrid coupler 248 may be set, such that C₁=99 pF, C₂=41 pF, and L=175 nH, at 3.0 Tesla, for example, at a carbon magnetic resonance frequency of 32.13 MHz.

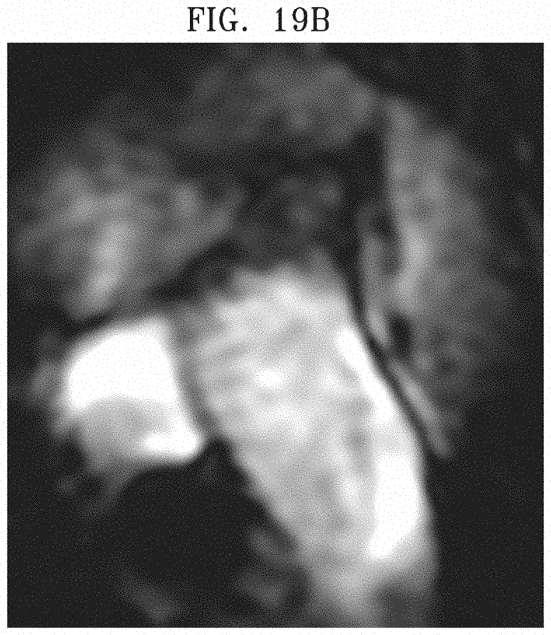

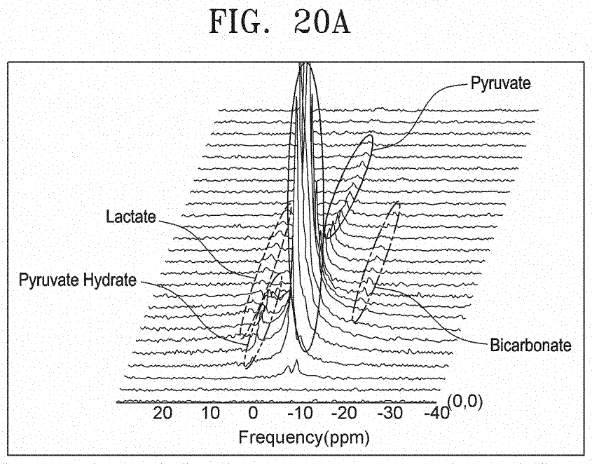

FIG. 15 is a circuit diagram illustrating a transmit/receive switching circuit in a radio frequency transmit/receive coil, according to an embodiment of the present disclosure. FIG. 16 is a circuit diagram illustrating that a one-channel radio frequency coil is connected to the transmit/receive switching circuit of FIG. 15.

Meanwhile, in the transmit/receive switching circuit 249, the values of ‘L’ and ‘C’ are selected as values matched to 50 ohms and delayed by ¼λ at a given frequency. In this case, a forward/reverse diode is shorted by a high-voltage signal in transmission, and is open with only a small magnetic resonance signal in reception.

Accordingly, a preamplifier for reception is protected from a higher power signal when a signal is transmitted. When a signal is received, a signal at a receiving stage is sent to a receive port and the connection to a transmit port is disconnected.

A circuit illustrated in FIG. 15 may be directly connected to a radio frequency amplifier. When a conventional simple connection box for the one-channel radio frequency coil is employed, the connection box may be used in the connection to the transmit/receive radio frequency switch circuit as in FIG. 15.

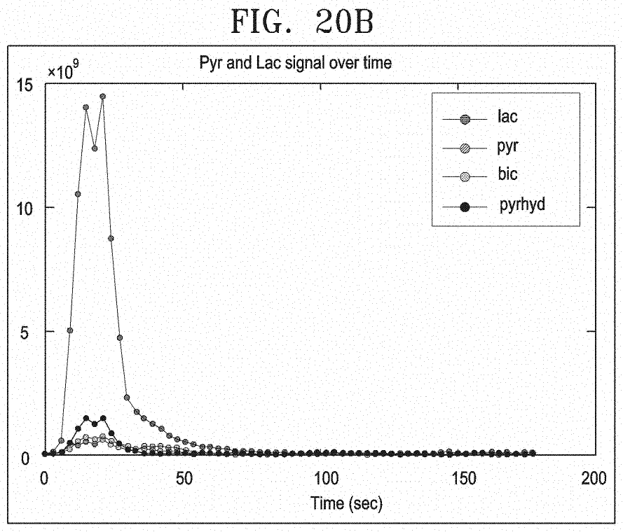

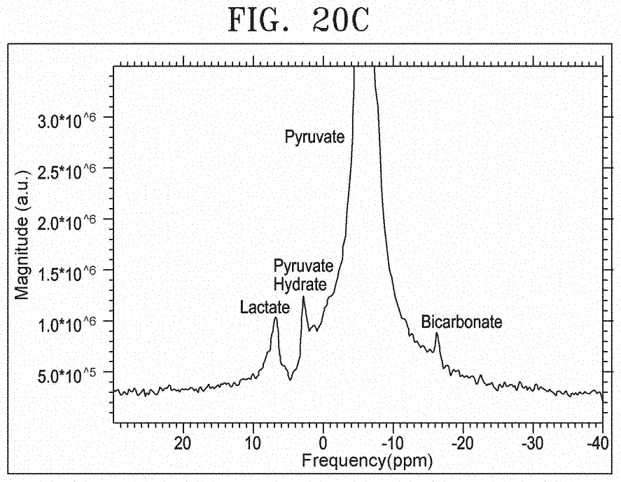

FIGS. 17 and 18 illustrate a reflection attenuation constant as a function of a frequency through tuning and matching to be matched to a frequency of a signal, through a radio frequency transmit/receive coil, according to an embodiment of the present disclosure;

Meanwhile, FIGS. 17 and 18 illustrate photographs obtained by actually capturing a reflection attenuation constant as a function of a frequency in a different nuclide coil manufactured according to the present disclosure. It may be recognized that a log scale shows 30.69 dB, and an impedance shows 50.7+j2.9 ohms in a Smith chart. Ideally, it may be recognized that the different nuclide coil is designed and manufactured such that a loss is minimized, as the impedance approaches 50 ohms at a different nuclide frequency.

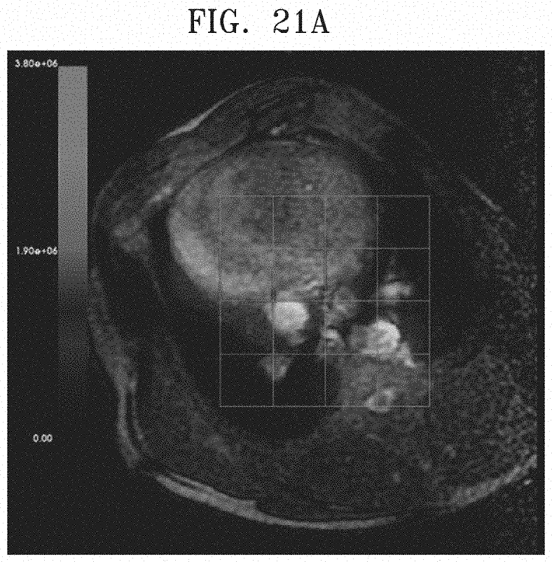

FIG. 19A is an image illustrating the magnetic resonance image of a swine heart captured using a hydrogen body coil, when a radio frequency transmit/receive coil is installed according to an embodiment of the present disclosure. FIG. 19B is an image illustrating a magnetic resonance image of a swine heart, when the radio frequency transmit/receive coil is not installed, according to an embodiment of the present disclosure.

In addition, referring to FIG. 19A and FIG. 19B, when compared with the image of FIG. 19B, the image of FIG. 19A shows an SNR reduced by about 8% in the area of the swine heart, which refers to that the open/short switching circuit properly operates to prevent the hydrogen magnetic resonance image from being damaged. A plurality of images may be acquired by merely adding and installing the second radio frequency transmit/receive coil unit 244 without changing the MRI system having a conventional hydrogen coil.

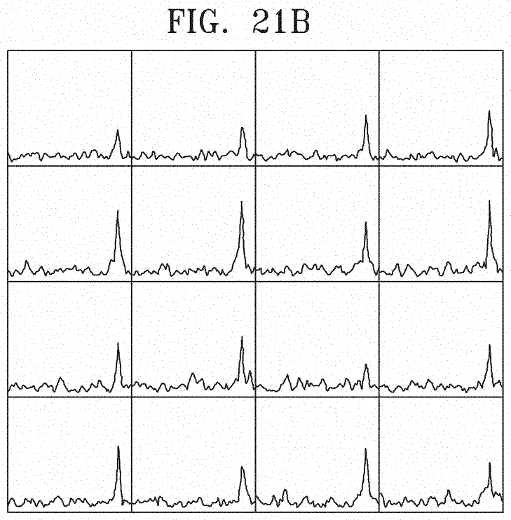

FIG. 20A is a graph showing a 13C dynamic magnetic resonance spectroscopy showing the results of pyruvate metabolism obtained a total of 60 times every 3 seconds according to an embodiment of the present disclosure. FIG. 20B is a graph showing magnetic resonance spectroscopy signals of pyruvate, lactic acid, bicarbonate, and pyruvate hydrate over time according to an embodiment of the present disclosure. FIG. 20C is a graph illustrating the summed spectrum of the 13C dynamic spectrum acquired from the result of FIG. 20A.

Referring to FIG. 20, the pyruvate, lactic acid, bicarbonate, and pyruvate hydrates are observed in the spectrum as a result of pyruvate metabolism, which serves as the result of the 13C dynamic magnetic resonance spectroscopy. Therefore, it may be recognized that the second radio frequency transmit/receive coil unit 244 is sufficiently operated at resonance frequency bands of the hydrogen and carbon 13.

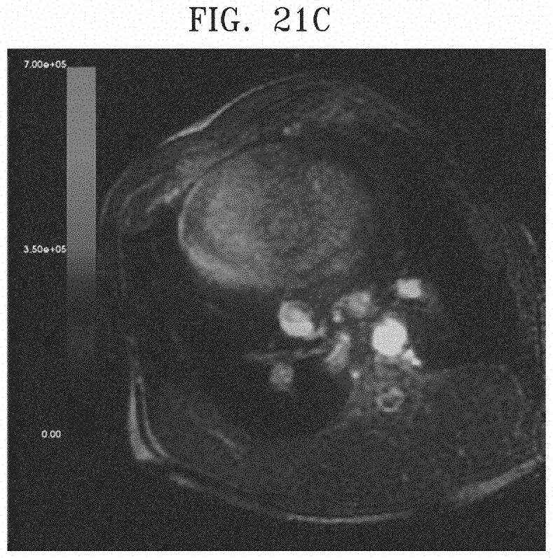

FIG. 21A illustrates a pseudo-color map of an experimental result of free-induction decay chemical shift imaging (FID-CSI) and a pyruvate signal in a 4×4 spectrum grid of the swine heart area, according to an embodiment of the present disclosure, FIG. 21B illustrates a 13C spectrum in the 4×4 spectrum grid, according to an embodiment of the present disclosure, FIG. 21C is an image representing a pseudo-color map of a lactic acid signal in a swine heart, according to an embodiment of the present disclosure.

Referring to FIGS. 21A to 21C, it may be recognized that the second radio frequency transmit/receive coil unit 244 smoothly operates at the 13-carbon resonance frequency. When pyruvate is injected into the artery of the swine, the result of pyruvate metabolism may be identified, based on the pseudo color map of pyruvate and lactic acid signals, through the second radio frequency transmit/receive coil unit 244.

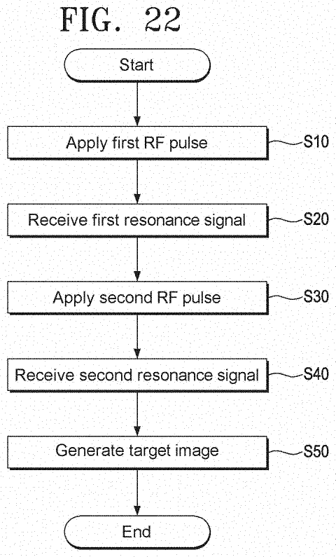

FIG. 22 is a flowchart illustrating a magnetic resonance imaging method, according to an embodiment of the present disclosure.

Referring to FIG. 22, according to an embodiment of the present disclosure, the magnetic resonance imaging method may include the steps of applying a first radio frequency pulse (S10), receiving a first resonance signal (S20), applying a second radio frequency pulse (S30), receiving a second resonance signal (S40), and generating a target image (S50).

In this case, the magnetic resonance imaging method includes steps processed in time-series in the magnetic resonance imaging system 100 as illustrated in FIGS. 1 and 2. Accordingly, it may be understood that the above description of the magnetic resonance imaging system 100 illustrated in FIGS. 1 and 2 may be applied to the magnetic resonance imaging method of FIG. 22, even if the details of the magnetic resonance imaging method will be omitted.

In addition, although not illustrated, before the step of applying a first radio frequency pulse (S10), there may be further included the step of additionally installing the second radio frequency transmit/receive coil unit 244 in the magnet device 240 including the main magnetic field coil unit 241, the gradient coil unit 242, the first radio frequency transmit/receive coil unit 243, and the second radio frequency transmit/receive coil unit 244.

In this case, the second radio frequency transmit/receive coil unit 244 may be interposed between the first radio frequency transmit/receive coil unit 243 and the target 10.

In the step of applying the first radio frequency pulse (S10), the first radio frequency pulse having the first frequency for exciting a first atomic nucleus of the target 10 is applied to the target 10 in the first radio frequency transmit/receive coil unit 243.

In this case, the second radio frequency transmit/receive coil unit 244 may be designed, such that a circuit is open at the first frequency, and shorted at a second frequency.

In other words, in the step of applying the first radio frequency pulse (S10), when the first radio frequency pulse having the first frequency is applied through the first radio frequency transmit/receive coil unit 243, the first frequency is applied to the target 10 through the second radio frequency transmit/receive coil unit 244. The second radio frequency transmit/receive coil unit 244 is electrically open in this process, such that an influence is not exerted on transmitting or receiving the first frequency.

In addition, in the step of receiving the first resonance signal (S20), the magnetic resonance imaging device 110 may receive a first magnetic resonance signal emitted by a radio frequency pulse and specific pulse sequences applied to the first atomic nucleus through the first radio frequency transmit/receive coil unit 243.

In addition, to acquire the image from the first atomic nucleus after receiving the image through a coil provided on the surface of the target with a higher an SNR, the first radio frequency transmit/receive coil is employed only in transmission, and the first radio frequency receive coil unit 245 is additionally employed to receive a resonance signal from the first atomic nucleus.

In addition, in the step of applying the second radio frequency pulse (S30), the second radio frequency pulse having the second frequency for exciting a second atomic nucleus of the target 10 is applied to the target 10 in the second radio frequency transmit/receive coil unit 244.

In this case, the second radio frequency transmit/receive coil unit 244 may be designed, such that a circuit is open at the first frequency, and shorted at the second frequency.

In addition, in the step of receiving the second resonance signal (S40), the magnetic resonance imaging device 110 may receive a second magnetic resonance signal emitted by a radio frequency pulse and specific pulse sequences applied to the second atomic nucleus through the second radio frequency transmit/receive coil unit 244.

In the step of generating a target image (S50), the image processing device 120 may generate an image of the target 10 using magnetic resonance signals sequentially received.

Accordingly, the magnetic resonance imaging device 110 may obtain both anatomical information and metabolic information of a biological subject, by generating a magnetic resonance image, based on magnetic resonance signals obtained using a plurality of different types of atomic nuclei, as well as one type of atomic nucleus.

The above description has been made for the illustrative purpose. Furthermore, the above-mentioned contents describe an embodiment of the present disclosure, and the present disclosure may be used in various other combinations, changes, and environments. That is, the present disclosure can be modified and corrected without departing from the scope of the present disclosure that is disclosed in the specification, the equivalent scope to the written disclosures, and/or the technical or knowledge range of those skilled in the art. The written embodiment describes the best state for implementing the technical spirit of the present disclosure, and various changes required in the detailed application fields and purposes of the present disclosure can be made. The written embodiment describes the best state for implementing the technical spirit of the present disclosure, and various changes required in the detailed application fields and purposes of the present disclosure can be made. Furthermore, it should be construed that the attached claims include other embodiments.

1. A radio frequency transmit/receive coil comprising:
a pair of end coils provided at an upper end and a lower end of the radio frequency transmit/receive coil and having a ring shape;
a plurality of leg coils to connect the pair of end coils to each other; and
switching module disposed between the pair of end coils and the plurality of leg coils,
wherein the switching module is open by a first frequency, and shorted by a second frequency different from the first frequency.

2. The radio frequency transmit/receive coil of claim 1, wherein the end coil includes:
an upper coil disposed at the upper and a lower coil disposed at the lower end,
wherein the upper coil and the lower coil includes:
at least one spacing area spaced in a circumference direction, and
wherein the switching module is disposed in the spacing area to connect the upper coil or the lower coil which is spaced.

3. The radio frequency transmit/receive coil of claim 2, wherein the switching module includes:
a parallel-resonance circuit including an inductor and a first capacitor; and
a second capacitor which is series-connected to the parallel-resonance circuit.

4. The radio frequency transmit/receive coil of claim 2, wherein the switching module include:
a parallel-resonance circuit including a capacitor and a first inductor; and
a second inductor which is series-connected to the parallel-resonance circuit.

5. The radio frequency transmit/receive coil of claim 1, wherein the first frequency is a resonance frequency for exciting a hydrogen atom nucleus, and
wherein the second frequency is a resonance frequency for exciting a non-hydrogen atom nucleus.

6. A magnetic resonance imaging (MRI) device comprising:
a processor configured to determine pulse sequences applied to a target placed inside a static magnetic field;
a first radio frequency transmit/receive coil unit configured to apply a first radio frequency pulse having a first frequency for exciting a first atomic nucleus included in the target, and receive a first magnetic resonance signal emitted by the first radio frequency pulse;
a second radio frequency transmit/receive coil unit configured to apply a second radio frequency pulse having a second frequency for exciting a second atomic nucleus included in the target and receive a second magnetic resonance signal emitted by the second radio frequency pulse; and
a signal acquiring unit configured to process the first magnetic resonance signal and the second magnetic resonance signal,
wherein an operation of the second radio frequency transit/receive coil unit is stopped, when the first radio frequency pulse is applied to the target through the first radio frequency transmit/receive coil unit.

7. The MRI device of claim 6, wherein the first radio frequency transmit/receive coil unit includes:
a switching module open at the first frequency, and shorted at the second frequency different from the first frequency.

8. The MRI device of claim 7, wherein the first radio frequency transmit/receive coil unit includes:
a first radio frequency transmit coil unit to apply the first radio frequency pulse having the first frequency for exciting the first atomic nucleus included in the target; and
a first radio frequency receive coil unit to receive the first magnetic resonance signal emitted by the first radio frequency pulse,
wherein the first radio frequency receive coil unit is interposed between the second radio frequency transmit/receive coil unit and the target, shorted at the first frequency, and open at the second frequency.

9. The MRI device of claim 6, wherein a switching module of the second radio frequency transmit/receive coil unit include:
a parallel-resonance circuit including an inductor and a first capacitor; and
a second capacitor which is series-connected to the parallel-resonance circuit.

10. The MRI device of claim 8, wherein the first frequency is a resonance frequency for exciting a hydrogen atomic nucleus, and
wherein the second frequency is a resonance frequency for exciting a non-hydrogen atomic nucleus.

11. The MRI device of claim 8, wherein the second radio frequency transmit/receive coil unit is interposed between the target and the first radio frequency transmit/receive coil unit.

12. A magnetic resonance imaging (MRI) method comprising:
(I) applying a first radio frequency pulse having a first frequency for exciting a first atomic nucleus included in a target placed in a static magnetic field;
(II) receiving a first magnetic resonance signal emitted by the first radio frequency pulse applied to the first atomic nucleus;
(III) applying a second radio frequency pulse having a second frequency for exciting a second atomic nucleus included in the target;
(IV) receiving a second magnetic resonance signal emitted by the second radio frequency applied to the second atomic nucleus; and
(V) generating an image of the target by using the first magnetic resonance signal and the second magnetic resonance signal.

13. The method of claim 12, wherein the (I) step and the (II) step are performed by a first radio frequency transmit/receive coil unit to apply the first radio frequency pulse having the first frequency for exciting the first atomic nucleus included in the target and to receive the first magnetic resonance signal emitted by the first frequency pulse,
wherein the (III) step and the (IV) step are performed by a second radio frequency transmit/receive coil unit to apply the second radio frequency pulse having the second frequency for exciting the second atomic nucleus included in the target and to receive the second magnetic resonance signal emitted by the second radio frequency pulse, and
wherein an operation of the second radio frequency transmit coil unit is stopped, when the first radio frequency pulse is applied to the target through the first radio frequency transmit/receive coil unit in the (I) step.

14. The method of claim 12, wherein the (I) step is performed by a first radio frequency transmit coil unit to apply the first radio frequency pulse having the first frequency for exciting the first atomic nucleus included in the target,
wherein the (II) step is performed by the first radio frequency receive coil unit to receive the first magnetic resonance signal emitted by the first frequency pulse,
wherein the (III) step and (IV) step are performed by a second radio frequency transmit/receive coil unit to apply the second radio frequency pulse having the second frequency for exciting the second atomic nucleus included in the target and to receive the second magnetic resonance signal emitted by the second radio frequency pulse,
wherein an operation of the second radio frequency transmit/receive coil unit and an operation of the first radio frequency receive coil unit are stopped, when the first radio frequency pulse is applied to the target through the first radio frequency transmit coil unit in the (I) step, and
wherein the operation of the first radio frequency receive coil unit is stopped, when the second radio frequency pulse is applied to the target through the second radio frequency transmit/receive coil unit in the (III) step.

15. The method of claim 13, wherein the second radio frequency transmit/receive coil unit includes:
a switching module, and
wherein the switching module of the second radio frequency transmit/receive coil unit is open by the first frequency in the (I) step, and shorted at the second frequency different from the first frequency in the (III) step.

16. The method of claim 15, wherein the switching module of the second radio frequency transmit/receive coil unit include:
a parallel-resonance circuit including an inductor and a first capacitor and a second capacitor which is series-connected to the parallel-resonance circuit.

17. The method of claim 14, wherein the first radio frequency receive coil unit includes:
a switching module,
wherein the switching module of the first radio frequency receive coil unit is shorted by the first frequency in the (I) step, and open by the second frequency different from the first frequency in the (III) step, and
wherein a switching module is open, when the first frequency is transmitted.

18. The method of claim 17, wherein the switching module of the first radio frequency receive coil unit includes:
a parallel-resonance circuit including a capacitor and a first inductor and a second inductor which is series-connected to the parallel-resonance circuit.

19. The method of claim 15, wherein the first frequency is a resonance frequency for exciting a hydrogen atomic nucleus in the step (I), and
wherein the second frequency is a resonance frequency for exciting a non-hydrogen atomic nucleus in the step (III).
